(19) United States
(12) Patent Application Publication
(10) Pub. No.: US 2013/0345590 A1
(43) Pub. Date: Dec. 26, 2013

(54) APPARATUS FOR QUANTIFYING EXPIRATORY AND INSPIRATORY AIRFLOW

(71) Applicants: Hartmut Schneider, Lutherville, MD (US); Jason Paul Kirkness, Baltimore, MD (US)

(72) Inventors: Hartmut Schneider, Lutherville, MD (US); Jason Paul Kirkness, Baltimore, MD (US)

(73) Assignee: The Johns Hopkins University, Baltimore, MD (US)

(21) Appl. No.: 14/004,082

(22) PCT Filed: Mar. 9, 2012
(86) PCT No.: PCT/US12/28562

§ 371 Date: Sep. 9, 2013

Related U.S. Application Data
(60) Provisional application No. 61/450,985, filed on Mar. 9, 2011.

(51) Int. Cl. A61B 5/087; A61B 5/00

(52) U.S. Cl. CPC A61B 5/087; A61B 5/746; A61B 5/742
USPC 600/538

(57) ABSTRACT

An apparatus for quantifying a user's expiratory and inspiratory airflow includes an air tube adapted to be sealed over at least one of the nose or mouth of a user, a pressure sensor configured to be selectively fluidly connected with one of the air tube or an ambient environment external to the air tube, a valve assembly arranged between the air tube and the pressure sensor to switch between a measuring configuration in which the pressure sensor is fluidly connected with the air tube while fluid connection with the ambient environment is excluded, and a reference configuration in which the pressure sensor is fluidly connected with the ambient environment while fluid connection with the air-tube is excluded, and a data processing unit arranged to communicate with the pressure sensor and the valve assembly. The data processing unit is configured to provide instructions to the valve assembly to switch between the measuring and the reference configurations. The data processing unit is further configured to determine an absolute zero of expiratory and inspiratory airflow based on signals from the pressure sensor obtained while the valve assembly is in the reference configuration and to measure at least one of expiratory and inspiratory airflow while the valve assembly is in the measuring configuration. The processing unit is further configured to determine at least one of expiratory airflow limitation or inspiratory airflow limitation relative to the absolute zero airflow.

### CROSS-REFERENCE OF RELATED APPLICATION

This application claims priority to U.S. Provisional Application No. 61/450,985 filed Mar. 9, 2011, the entire contents of which are hereby incorporated by reference.

This invention was made with U.S. Government support of Grant No. P50 HL084945, awarded by SCCOR. The U.S. Government has certain rights in this invention.

### BACKGROUND

1. Field of Invention

The field of the currently claimed embodiments of this invention relates to apparatuses and methods for quantifying inspiratory and expiratory airflow and characterizing respiratory disorders.

2. Discussion of Related Art

The gold standard assessment of ventilation is the measurement of airflow with a pneumotachograph (American Sleep Disorders Assoc. 1995; Kushida et al. 2005; American Academy of Sleep Medicine 1999), although it is not routinely used, instead semi-quantitative measures of airflow are utilized. Pneumotachographs measure airflow through a tube that imposes a small resistance to airflow, which is not a problem during wakefulness in normal healthy individuals. During sleep or in patients at risk for respiratory failure airflow, the added resistance or any deadspace is a problem primarily for two reasons. First, the added resistance might change respiratory pattern and ventilation particularly when flow is at a minimum (Hlavac et al. 2007; Morrell, Browne, & Adams 2000 ; Pillar et al. 2000; Tun et al. 2000; Hudgel, Mulholland, & Hendricks 1987). Second, the use of a pneumotachograph may exacerbate respiratory failure in patients who cannot adapt their respiratory pattern in response to the added load. Another reason why pneumotachographs are rarely used in clinical practice during sleep.

In addition, measuring inspiratory and expiratory airflow limitation requires simultaneous measures of airflow and airway pressures, because of uncertainties of the absolute zero of airflow measurements. Currently, defining the absolute zero is a major problem in measuring airflow since both electrical and mechanical signals drift over time, leading to inaccuracies of measuring airflow. Current methods of defining the absolute zero use either software or hardware algorithms that can have at least two disadvantages: 1) Software algorithms distort the airflow signal thereby affecting the airflow contour; and 2) Hardware algorithms can detect the absolute zero but they do not correct the electrical or mechanical drifts in the airflow signal. There thus remains a need for improved apparatuses for quantifying respiratory and inspiratory airflow.

### SUMMARY

An apparatus for quantifying a user's expiratory and inspiratory airflow according to an embodiment of the current invention includes an air tube adapted to be sealed over at least one of the nose or mouth of a user, a pressure sensor configured to be selectively fluidly connected with one of the air tube or an ambient environment external to the air tube, a valve assembly arranged between the air tube and the pressure sensor to switch between a measuring configuration in which the pressure sensor is fluidly connected with the air tube while fluid connection with the ambient environment is excluded, and a reference configuration in which the pressure sensor is fluidly connected with the ambient environment while fluid connection with the air-tube is excluded, and a data processing unit arranged to communicate with the pressure sensor and the valve assembly. The data processing unit is configured to provide instructions to the valve assembly to switch between the measuring and the reference configurations. The data processing unit is further configured to determine an absolute zero of expiratory and inspiratory airflow based on signals from the pressure sensor obtained while the valve assembly is in the reference configuration and to measure at least one of expiratory and inspiratory airflow while the valve assembly is in the measuring configuration. The processing unit is further configured to determine at least one of expiratory airflow limitation or inspiratory airflow limitation relative to the absolute zero airflow.

A method of quantifying a subject's respiratory and inspiratory airflow according to an embodiment of the current invention includes measuring at least one of respiratory airflow or inspiratory airflow of the subject, measuring an absolute zero airflow in a local environment of the subject, and determining at least one of expiratory airflow limitation or inspiratory airflow limitation based on the measuring at least one of respiratory airflow or inspiratory airflow relative to the absolute zero airflow.

### BRIEF DESCRIPTION OF THE DRAWINGS

Further objectives and advantages will become apparent from a consideration of the description, drawings, and examples.

FIG. 1 is a schematic illustration of an apparatus for quantifying respiratory and inspiratory airflow according to an embodiment of the current invention.

FIGS. 2A-2C provide front, cross-section and perspective views of a portion of illustrate the apparatus of FIG. 1.

FIG. 3 is a schematic illustration of an apparatus for quantifying respiratory and inspiratory airflow according to an embodiment of the current invention.

FIG. 4 is an illustration of a differential pressure transducer according to an embodiment of the current invention.

FIG. 5 is a schematic illustration of a portion of a valve assembly of the apparatus of FIG. 1.

FIG. 6 shows an example of data according to an embodiment of the current invention.

FIG. 7 shows examples of breath contours to explain the operation of an apparatus according to some embodiments of the current invention.

FIG. 8 is an example of actual data according to an embodiment of the current invention to illustrate periods of normal ventilation and dynamic hyperinflation.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Some embodiments of the current invention provide methods and devices that allows quantifying on a breath by breath basis the degree of inspiratory and expiratory flow limitation and dynamic hyperinflation. As described in more detail below, this approach utilizes the absolute zero and deviation in measured airflow at specific time points from the zero line. The degree of inspiratory airflow limitation can provide a marker for the degree of upper airway obstruction and can be obtained by measuring the level of airflow during inspiration. Similarly, by using the expiratory flow contour some embodiments of the current invention allows one to determine the degree of expiratory airflow limitation and magnitude of dynamic hyperinflation, both of which are hallmarks for the severity of asthma and chronic obstructive lung disease. Current airflow sensors miss these markers of inspiratory upper airway obstruction, COPD and Asthma.

Some embodiments of the current invention obviate measuring airway pressures by determining the absolute zero from the airflow signal. Thus, some embodiments of the current invention allow for the quantification of inspiratory and expiratory airflow limitation and dynamic hyperinflation from the airflow signal alone. An apparatus according to an embodiment of the current invention measures repeatedly the absolute zero and corrects electrical and mechanical drifts.

Some embodiments of the current invention can solve several problems. First, it defines absolute zero without distorting the airflow signal and it can automatically correct the airflow signal based on the measured absolute zero. Second, by knowing the absolute zero, the new apparatus would also prevent an overestimation and/or underestimation of inspiratory airflow, which accrue in existing methods due to the problems mentioned above. Third, the repeated re-zeroing of any electrical drift and automated calibration by referencing the airflow to atmosphere allows accurate airflow measurements over limitless time periods. Therefore, embodiments of the current invention are suited to accurately quantify and monitor inspiratory and expiratory disorders of breathing.

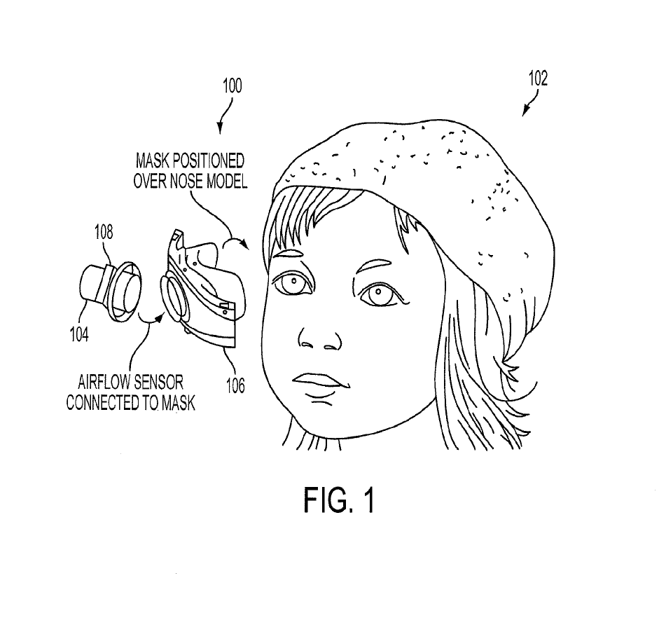

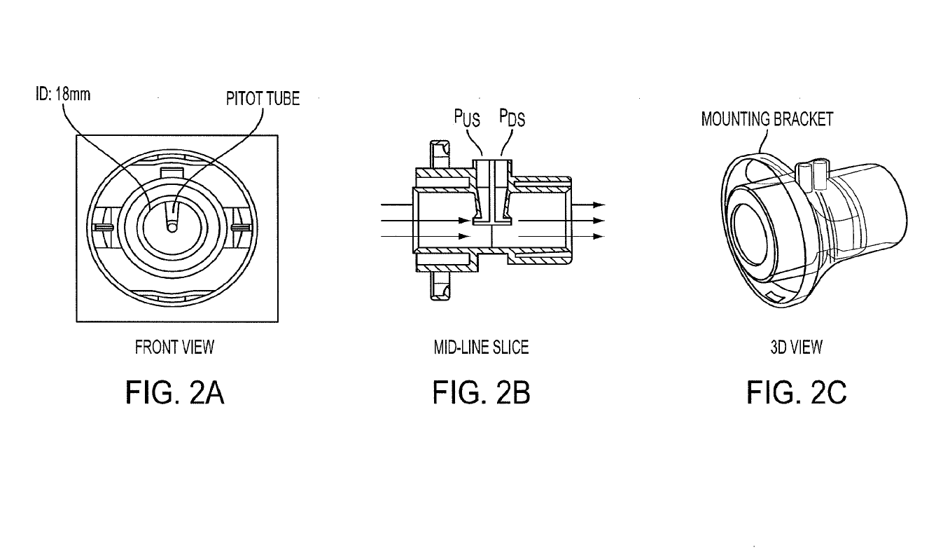

FIG. 1 provides a schematic illustration of an apparatus 100 for quantifying a user's 102 respiratory and inspiratory airflow according to an embodiment of the current invention. The apparatus 100 has an air tube 104 adapted to be sealed over at least one of the nose or mouth of a user 102. The air tube 104 can be a separate component that can be attached and removed from a mask 106 in some embodiments of the current invention. In alternative embodiments, the air tube 104 and mask 106 can be integral as a single unit. In some embodiments, the air tube 104 can be sealed over the nose of the user 102 with the mask 106, as illustrated in the example of FIG. 1. In alternative embodiments, the air tube 104 can be sealed over the mouth, or over the nose and mouth of the user 102. The air tube 104 can include a Pitot tube 108 to be attached to a pressure sensor. Also, see FIGS. 2A-2C for more detailed illustrations of an example of air tube 104.

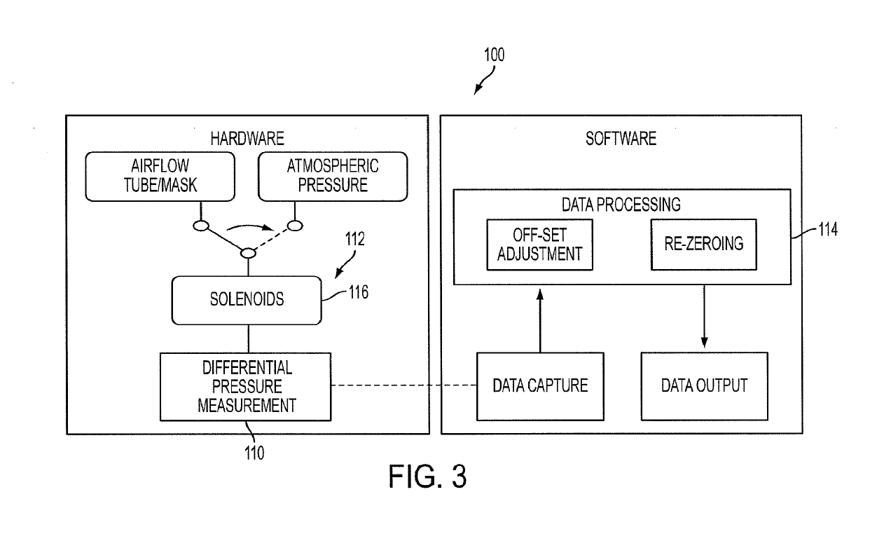

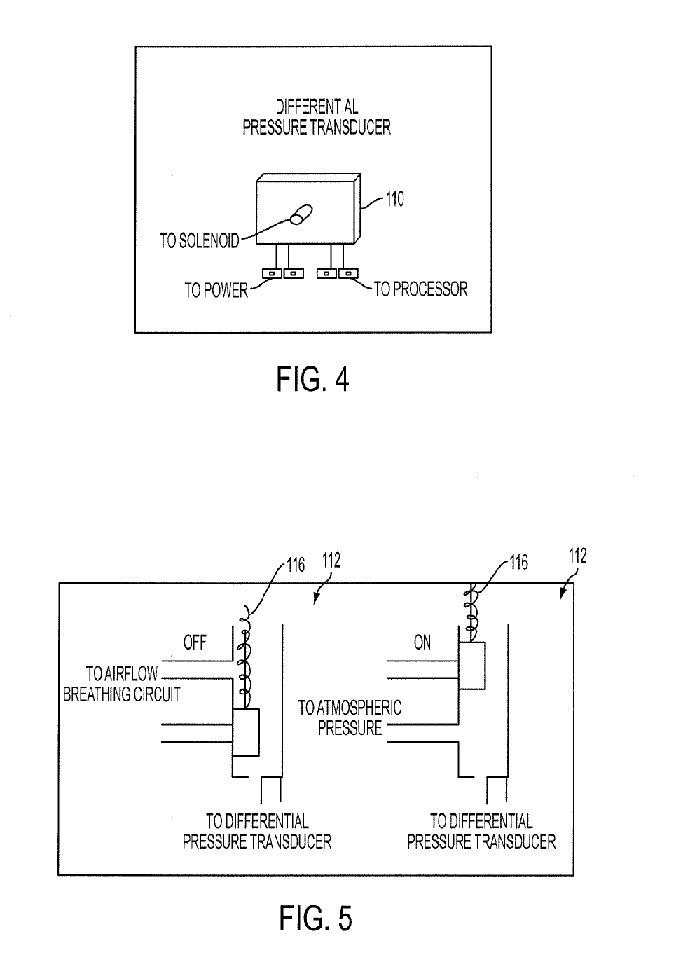

FIG. 3 is a schematic illustration of the apparatus 100, including software, according to an embodiment of the current invention. The apparatus 100 also include a pressure sensor 110 configured to be selectively fluidly connected with one of the air tube 104 or an ambient environment (atmospheric pressure) external to the air tube 104. The pressure sensor can be a differential pressure transducer, for example (see, also, FIG. 4).

The apparatus 100 further includes a valve assembly 112 arranged between the air tube 104 and the pressure sensor 110 to switch between a measuring configuration in which the pressure sensor 110 is fluidly connected with the air tube 104 while fluid connection with the ambient environment is excluded, and a reference configuration in which the pressure sensor 110 is fluidly connected with the ambient environment while fluid connection with the air tube 104 is excluded. The apparatus 100 further includes a data processing unit 114 arranged to communicate with the pressure sensor 110 and the valve assembly 112. The data processing unit 114 is configured to provide instructions to the valve assembly 112 to switch between the measuring and the reference configurations. The data processing unit 114 is further configured to determine an absolute zero of respiratory and inspiratory airflow based on signals from the pressure sensor 110 obtained while the valve assembly is in the reference configuration and to determine a net difference in respiratory and inspiratory flow with respect to the absolute zero. The valve assembly 112 can include a solenoid actuator 116 for switching the valve between the measuring and reference configurations. (See, also, FIG. 5.)

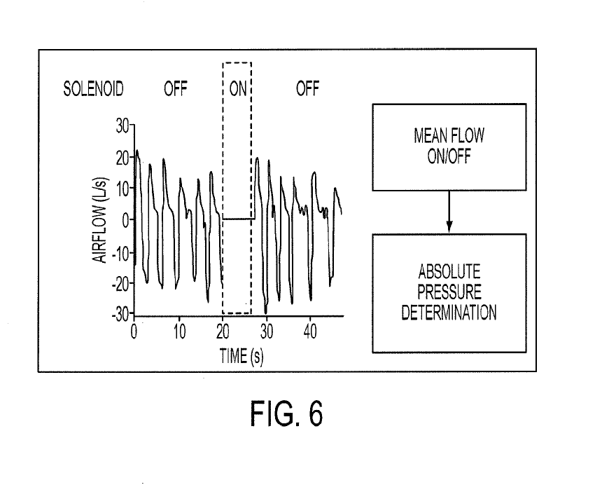

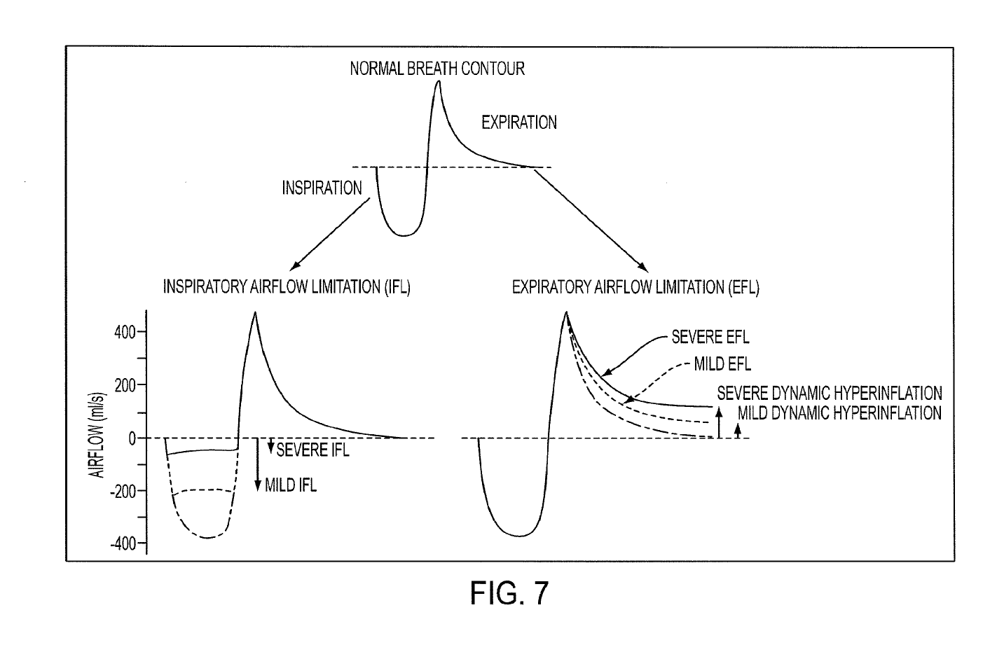

FIG. 6 shows an example of measured airflow over a period of time that includes a dozen breaths. The portion of the curve during the “ON” state is the reference configuration in which the absolute zero was being determined.

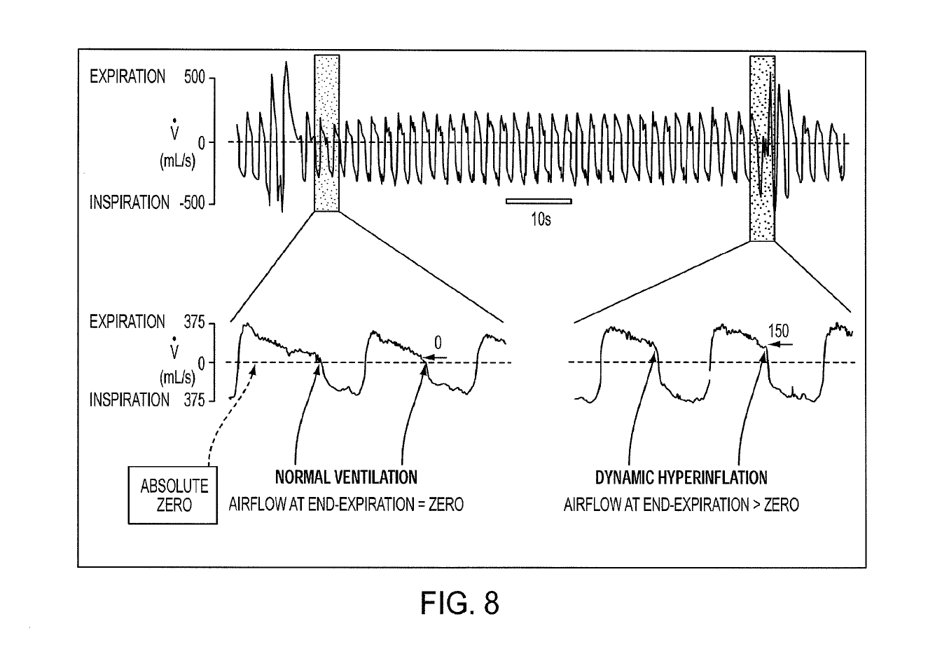

FIG. 7 is a schematic illustration of one inspiration-expiration cycle shown in more detail. The top-center diagram shows a normal breath contour. The dashed line is the absolute zero, as determined for this case. The lower-left diagram in FIG. 7 illustrates detection and assessment of inspiratory airflow limitation (IFL) according to an embodiment of the current invention. In this case, the shape of the inspiration phase is flatter than the normal contour. In addition, by determining the absolute zero (horizontal dashed line at zero airflow), the severity of the IFL can also be quantified. The lower-right diagram in FIG. 7 illustrates detection and assessment of expiratory airflow limitation (EFL) according to an embodiment of the current invention. Since the dashed horizontal line is a measured absolute zero in the airflow, the offset in the asymptotic decay of the expiration phase can be detected and quantified. One can see that if the absolute zero in airflow had not been measured, one would not know whether the location of the horizontal dashed line was correct, and in fact could be arbitrarily shifted up or down. Therefore, by measuring the absolute zero of airflow, EFL can be detected and quantified. In addition, since airflow is movement of air as over time, the contours can be integrated over time to determine a net, non-zero flow. FIG. 8 shows an actual data taken with an apparatus according to an embodiment of the current invention. In this example, the user has periods of normal ventilation, followed by a period of dynamic hyperinflation.

In some embodiments of the current invention, the data processing unit 114 can be further configured to output information to a user-output-component based on the net difference in respiratory and inspiratory flow with respect to the absolute zero. In some embodiments, the user-output-component can include at least one of an audio or video alarm, for example. In some embodiments, the user-output-component can include a video display adapted to display at least one of alphanumeric or graphical information, for example.

In some embodiments, the apparatus 100 can further include a data storage unit in communication with the data processing unit 114. The data storage unit can be adapted to store at least one of signals from the pressure sensor or calculated values from the data processing unit for later retrieval. The data storage unit can include a removable data storage medium, for example. In some embodiments, the apparatus 100 can further include a data interface to at least retrieve data stored in the data storage unit.

The apparatus according to some embodiments of the current invention can provide solutions for detecting inspiratory and expiratory flow limitation and dynamic hyperinflation, for example. In an embodiment, the apparatus has four parts (see FIG. 3): 1) a pressure measuring unit, 2) solenoids, 3) electrical relays, and 4) an electrical processor unit that houses hardware and software algorithms. The pressure measuring unit can be standard pressure transducers. The solenoids are designed to disconnect the pressure measuring unit from the patient and open the pressure transducers to atmosphere, which defines the absolute zero for breathing. The relays can include a software and hardware algorithm that periodically switches the solenoids. The electrical processor unit uses the simultaneous measurement of atmospheric pressure to provide repeated re-zeroing of any electrical drift and automated calibration. This has been an unsolved problem of current technologies.

### Pitot Flowmeter

In an example, the ‘Pitot flowmeter’ is a polyethylene lightweight (1.5 grams), low dead-space (˜10 cm³) flowmeter (KeyFlow™, Key Technologies Inc, Baltimore, USA) that uses the Pitot tube principal to determine midstream airflow rate flowing through a wide bore flow tube (FIG. 2). The flow sensor has two ports for pressure measurement located in the centerline of the flow tube; one oriented upstream (P_{US}; pressure head) and one oriented downstream (P_{DS}; tail pressure). The Pitot tube openings are positioned in line with airflow and detect the pressure head (rather than the side-stream pressure as in a pneumotachograph) of the bulk flow through the Pitot flowmeter. Both of the P_{US} and P_{DS} ports are connected via separate plastic tubing to either side of a differential pressure transducer that is mounted on a circuit board to amplify the signal. An algorithm converts the differential between the head and the tail pressure differential into a voltage output as is described below. The zero output of the Pitot flowmeter is set to 2.5 volts and the output range is from 0 to 5 volt in a particular example. The general concepts of the current invention are not limited to the particular examples.

### Airflow Measurement Principle

The major technical difference of the Pitot flowmeter to standard pneumotachograhs is that it measures midstream airflow rather than side stream pressure. The theoretical principle used in the Pitot flowmeter's measurement of airflow is derived from application of the Pitot tube approach and is based on the Bernoulli Equation:

Δp+ 1/2ρV²+ρgh=constant equation 1

where: Δp=differential pressure, ρ=density, V=velocity, g=gravity, h=elevation. This ideal equation is valid for measures at any point along a stream line for steady fluid flows with constant density and for which friction is negligible. Furthermore, both gravity and elevation are also constant and negligible in this application of equation 1. The differential pressure measured between the Pitot tube ports relates to the fluid velocity as follows:

pᵤₚ−p_{dn}=½ρV² equation 2

where, V is the velocity in the centerline of the flow sensor. The airflow rate is then calculated from the centerline velocity as follows:

Q=C·V·A equation 3

where, Q=flow rate, A=cross sectional area of the flow sensor, C=velocity profile pressure head correction factor in the flow sensor. During turbulent flow, the velocity profile of the pressure head is effectively flat, thus C is very close to 1.0. In fully developed laminar flow, the velocity profile is parabolic and C is closer to 0.5. In the Pitot flowmeter, the flow rate algorithm is empirically determined to account for variation of the velocity profile for changes in flow rate.

An apparatus and methods according to some embodiments of the current invention can allow for quantification of inspiratory and expiratory airflow for extended time periods without performing repeated manual calibration or correction procedures. By knowing the absolute zero, one can detect the magnitude of inspiratory and expiratory airflow limitation and the degree of dynamic hyperinflation. Dynamic hyperinflation occurs when inspiration starts prematurely while expiratory airflow is still present. The knowledge of an absolute zero can discriminate whether airflow immediately prior to an inspiration has ceased (e.g. it approaches zero) or not (e.g. if it exceeds the zero) (FIGS. 7 and 8). In patients with Asthma and COPD, dynamic hyperinflation is an indicator for disease severity. Thus, the new apparatus could be used as a monitor for Asthma and COPD disease severity, particularly during sleep.

Apparatuses according to some embodiments of the current invention can be applied in clinical or institutional settings and in the home environment for patients and subjects who need monitoring of airflow for diagnostic and therapeutic purposes or to control the efficacy of a given treatment that would affect ventilation.

Some applications can include the following:
Inspiratory Airflow Monitor: Snoring and sleep apnea are caused by upper airway collapse. The severity of upper airway collapse can be determined by the degree of inspiratory airflow limitation. The knowledge of an absolute zero allows quantifying the degree of inspiratory airflow limitation and thereby the degree of the underlying disturbance that causes sleep apnea and snoring. Quantifying upper airway properties by the inspiratory airflow monitor could be used to identify patients at risk for developing sleep apnea (like a blood pressure monitor detects the risk for developing stroke and heart failure) and it may be used to guide treatment for snoring and sleep apnea. Such a monitor would also allow detecting beneficial or adverse effects of therapeutic or non-therapeutic agents on upper airway properties.
Expiratory Airflow Monitoring: Breathing mechanics often worsens during sleep, sedation and anesthesia compared to wakefulness. In conjunction with a portable Air-Flow-meter device, the new method and apparatus would allow the detection of expiratory flow limitation and dynamic hyperinflation as a marker for the severity of asthma, COPD and emphysema. Thus, it could be used to monitor asthma and COPD severity during sleep, sedation or anesthesia and the effect of pharmacological and other treatments on the Asthma and COPD severity.

The magnitude of inspiratory and expiratory airflow limitation and the degree of dynamic hyperinflation can be determined on a breath-by-breath basis independently of additional pressure measurements.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. An apparatus for quantifying a user's expiratory and inspiratory airflow, comprising:
an air tube adapted to be sealed over at least one of the nose or mouth of a user;
a pressure sensor configured to be selectively fluidly connected with one of said air tube or an ambient environment external to said air tube;
a valve assembly arranged between said air tube and said pressure sensor to switch between a measuring configuration in which said pressure sensor is fluidly connected with said air tube while fluid connection with said ambient environment is excluded, and a reference configuration in which said pressure sensor is fluidly connected with said ambient environment while fluid connection with said air-tube is excluded; and
a data processing unit arranged to communicate with said pressure sensor and said valve assembly,
wherein said data processing unit is configured to provide instructions to said valve assembly to switch between said measuring and said reference configurations, and
wherein said data processing unit is configured to determine an absolute zero of expiratory and inspiratory airflow based on signals from said pressure sensor obtained while said valve assembly is in said reference configuration and to measure at least one of expiratory and inspiratory airflow while said valve assembly is in said measuring configuration, said processing unit being further configured to determine at least one of expiratory airflow limitation or inspiratory airflow limitation relative to said absolute zero airflow.

2. The apparatus according to claim 1, wherein said pressure sensor is a differential pressure sensor.

3. The apparatus according to claim 2, wherein said pressure sensor is selectively fluidly connected to said air tube through a Pitot tube.

4. The apparatus according to claim 3, wherein said valve assembly comprises a solenoid actuator.

5. The apparatus according to claim 1, wherein said data processing unit is further configured to output information to a user-output-component based on said determining at least one of expiratory airflow limitation or inspiratory airflow limitation relative to said absolute zero airflow.

6. The apparatus according to claim 5, wherein said user-output-component comprises at least one of an audio or video alarm.

7. The apparatus according to claim 5, wherein said user-output-component comprises a video display adapted to display at least one of alphanumeric or graphical information.

8. The apparatus according to claim 1, wherein said data processing unit is further configured to determine a degree of said at least one of expiratory airflow limitation or inspiratory airflow limitation based on an amount of offset from said absolute zero airflow.

9. The apparatus according to claim 1, wherein said data processing unit is further configured to determine a cumulative expiratory airflow limitation or inspiratory airflow limitation integrated over a plurality of breaths of said user.

10. The apparatus according to claim 1, further comprising a data storage unit in communication with said data processing unit, said data storage unit being adapted to store at least one of signals from said pressure sensor or calculated values from said data processing unit for later retrieval.

11. The apparatus according to claim 10, wherein said data storage unit comprises a removable data storage medium.

12. The apparatus according to claim 10, further comprising a data interface to at least retrieve data stored in said data storage unit.

13. A method of quantifying a subject's respiratory and inspiratory airflow, comprising:
measuring at least one of respiratory airflow or inspiratory airflow of said subject;
measuring an absolute zero airflow in a local environment of said subject; and
determining at least one of expiratory airflow limitation or inspiratory airflow limitation based on said measuring at least one of respiratory airflow or inspiratory airflow relative to said absolute zero airflow.

14. The method of claim 13, wherein said determining at least one of expiratory airflow limitation or inspiratory airflow limitation determines a degree of said at least one of expiratory airflow limitation or inspiratory airflow limitation based on an amount of offset from said absolute zero airflow.

15. The method of claim 13, wherein said determining at least one of expiratory airflow limitation or inspiratory airflow limitation determines a cumulative expiratory airflow limitation or inspiratory airflow limitation integrated over a plurality of breaths of said subject.
